(19) United States
(12) Patent Application Publication
(10) Pub. No.: US 2016/0270845 A1
(43) Pub. Date: Sep. 22, 2016

(54) CORONARY SINUS MITRAL ISTHMUS ABLATION CATHETER

(71) Applicant: Medtronic, Inc., Minneapolis, MN (US)

(72) Inventors: Mark A. BENSCOTER, Rochester, MN (US); Timothy G. LASKE, Shoreview, MN (US)

(21) Appl. No.: 14/662,977

(22) Filed: Mar. 19, 2015

(51) Int. Cl. A61B 18/14; A61B 19/00; A61B 17/12

(52) U.S. Cl. CPC A61B 18/1492; A61B 17/1204; A61B 17/12136; A61B 19/54; A61B 2019/5466; A61B 2018/00351

(57) ABSTRACT

A device, system and method for creating transmural lesions between the coronary sinus and left atrium. The device includes an elongate body that is deflectable in two locations to create a transverse portion that is substantially orthogonal to the longitudinal axis of the elongate body and a distal tip portion that defines a longitudinal axis that is parallel to the longitudinal axis of the elongate body. The device may also include two electrodes, an occlusion balloon, a hemisphere marker, and a magnet in the distal portion. In use, one device may be positioned in the coronary sinus and another device may be placed in the left atrium proximate the mitral valve, the magnets being attracted to each other and magnetically coupling the two devices against tissue, through which a transmural lesion may be created when energy is delivered from at least one of the two electrodes of each device.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a method, system, and device for creating transmural ablation lesions and performing sensing functions in the coronary sinus and proximate the mitral isthmus.

### BACKGROUND OF THE INVENTION

Atrial fibrillation is a common cardiac condition in which abnormal electrical impulses within the heart tissue cause abnormal heart rhythm. Episodes of atrial fibrillation may be temporary, lasting from a few minutes to a few days (paroxysmal atrial fibrillation) or the condition may be permanent (persistent atrial fibrillation). These abnormal impulses may originate from the pulmonary veins, and pulmonary vein isolation is typically used to disrupt the conduction of abnormal electrical impulses to restore the heart's normal rhythm. However, pulmonary vein isolation is not curative in all patients with paroxysmal atrial fibrillation and in the majority of patients with persistent atrial fibrillation.

Ablation of the mitral isthmus can be conducted within the coronary sinus, from the coronary sinus to the mitral isthmus, and on the left atrial mitral isthmus endocardium. These ablation techniques have been found to have a positive effect on both paroxysmal and persistent types of atrial fibrillation, especially when used in conjunction with pulmonary vein isolation. In addition, some patients experience atrial flutter that involves the atrial tissue surrounding the mitral valve annulus. This mitral isthmus-dependent atrial flutter may be related to an ongoing disease process within the atrium, or can be secondary to an ongoing or prior cardiac ablation. To terminate such a flutter, a transmural line of conduction block needs to be created on the mitral valve annulus, spanning through the coronary sinus. Ablation of the tissues adjacent to the coronary sinus may be performed from within the coronary sinus or from the endocardial aspect of the mitral valve annulus. To be effective, however, the ablation typically requires transmural lesions spanning the entire myocardial thickness. This is often difficult because the ablation catheter may not be easily located within the coronary sinus, may not be positioned close enough to the mitral valve annulus, there may be fat adjacent to the coronary sinus, the catheter may be unable to maintain sufficient tissue contact, and/or may not deliver enough ablation energy (for example, to prevent collateral damage of non-target tissue). The anatomy of the coronary sinus varies in size and shape which impacts the usefulness of a catheter in its ability to maintain contact with the tissue.

It is therefore desirable to provide a method, system, and device that increase the safety and efficiency of ablation treatment for these forms of atrial fibrillation and atrial flutter. Specifically, it is desirable to provide a method, system, and device that is easily located and visualized at a target treatment location, has the ability to maintain position and tissue contact, is configured to precisely deliver energy at the treatment location, and that creates transmural lesions.

### SUMMARY OF THE INVENTION

The present application advantageously provides a method and system for creating transmural lesions between the coronary sinus and left atrial mitral isthmus. An ablation device may include an elongate body defining a longitudinal axis and having a distal portion, a proximal portion, a first deflection area in the distal portion, and a second deflection area in the distal portion, at least one ablation electrode at the distal portion, and a magnet at the distal portion. The ablation device may also include a first electrode, such as a distal tip electrode, and a second electrode, such as a band electrode. The band electrode may be located proximal to the distal tip electrode. The ablation device may also include a radiopaque marker, such as one that covers only a portion of a circumference of the elongate body. For example, the radiopaque marker may cover approximately half the circumference of the elongate body. The ablation device may further include an occlusion element coupled to the distal portion of the elongate body, and the occlusion device may be coupled to the distal portion of the elongate body at a location proximal to the first and second deflection areas. The elongate body may be transitionable between an at least substantially linear first configuration and a second configuration in which the distal portion of the elongate body includes a transverse portion that is substantially orthogonal to the longitudinal axis of the elongate body and a distal tip portion that defines a longitudinal axis that is non-coaxial with and parallel to the longitudinal axis of the elongate body. The transverse portion may define a longitudinal axis, the first deflection area being deflected approximately 90° from the longitudinal axis of the elongate body and the second deflection areas being deflected approximately 90° from the longitudinal axis of the transverse portion when the elongate body is in the second configuration. The magnet may be located within the distal portion of the elongate body proximate the band electrode.

An ablation system may include a first ablation device and a second ablation device, each of the first and second ablation devices including: an elongate body defining a distal tip and a longitudinal axis and having a distal portion, a proximal portion, a first deflection area in the distal portion, and a second deflection area in the distal portion; a tip ablation electrode at the elongate body distal tip and a band ablation electrode proximal to the tip electrode; a radiopaque marker covering approximately half a circumference of the elongate body and being located between the tip electrode and the band ablation electrode; and a magnet at the distal portion, the magnet of the first ablation device and the magnet of the second ablation device being configured to magnetically couple the first and second ablation devices through tissue, the first ablation device further including an occlusion balloon at the distal portion proximal to the at least one ablation electrode; and a control unit including an energy source in electrical communication with the at least one ablation electrode. The system may further include a fluoroscopic imaging system in communication with the control unit. The elongate body of the first ablation device may include a fluid injection lumen extending between the proximal portion of the elongate body to the distal portion of the elongate body, the distal tip defining a lumen opening. Further, the elongate body of each of the first and second ablation devices may be transitionable between an at least substantially linear first configuration and a second configuration in which the distal portion of the elongate body includes a transverse portion that is substantially orthogonal to the longitudinal axis of the elongate body and a distal tip portion that defines a longitudinal axis that is non-coaxial with and parallel to the longitudinal axis of the elongate body. The transverse portion may define a longitudinal axis, the first deflection area may be deflected approximately 90° from the longitudinal axis of the elongate body and the second deflection areas may be deflected approximately 90° from the longitudinal axis of the transverse portion when the elongate body is in the second configuration.

A method of treating cardiac arrhythmia may include: positioning a first ablation catheter in a coronary sinus proximate an endocardial left atrial mitral isthmus of a heart, the first ablation catheter including: an elongate body defining a distal tip and a longitudinal axis and having a distal portion, a proximal portion, a first deflection area in the distal portion, and a second deflection area in the distal portion; at least one ablation electrode on the distal portion of the elongate body; an occlusion balloon on the distal portion proximal to the first deflection portion; and a magnet at the distal portion distal to the second deflection portion; positioning a second ablation catheter within a left atrium of the heart proximate the endocardial left atrial mitral isthmus, the second ablation catheter including: an elongate body defining a distal tip and a longitudinal axis and having a distal portion, a proximal portion, a first deflection area in the distal portion, and a second deflection area in the distal portion; at least one ablation electrode on the distal portion of the elongate body; and a magnet at the distal portion distal to the second deflection portion, the magnet of the second ablation catheter being proximate the magnet of the first ablation catheter; magnetically coupling the first and second ablation catheters through tissue of the coronary sinus and left atrium; delivering radiofrequency energy to the at least one electrode of each of the first and second ablation catheters; and creating a transmural lesion in the tissue between the at least one electrode of the first ablation catheter and the at least one electrode of the second ablation catheter. The radiofrequency energy may be delivered in unipolar mode. The elongate body of each of the first and second ablation catheters may be transitionable between an at least substantially linear first configuration and a second configuration in which the distal portion of the elongate body includes a transverse portion that is substantially orthogonal to the longitudinal axis of the elongate body and a distal tip portion that defines a longitudinal axis that is non-coaxial with and parallel to the longitudinal axis of the elongate body.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows an anterior view of a human heart;

FIG. 2 shows a posterior view of a human heart;

FIG. 3 shows a top view of a human heart;

FIG. 4A shows an ablation system in accordance with the present invention, the system including one ablation catheter;

FIG. 4B shows an ablation system in accordance with the present invention, the system including two ablation catheters;

FIG. 5A shows a first embodiment of an ablation catheter in accordance with the present invention;

FIG. 5B shows a second embodiment of an ablation catheter in accordance with the present invention;

FIG. 6A shows a first ablation catheter and a second ablation catheter positioned proximate each other on opposite sides of cardiac tissue to create a transmural lesion;

FIG. 6B show a first ablation catheter and a second ablation catheter positioned proximate each other on opposite sides of cardiac tissue to create a transmural lesion and two additional lesions;

FIGS. 7-9 show a method of positioning the first ablation catheter and second ablation catheter proximate each other.

### DETAILED DESCRIPTION OF THE INVENTION

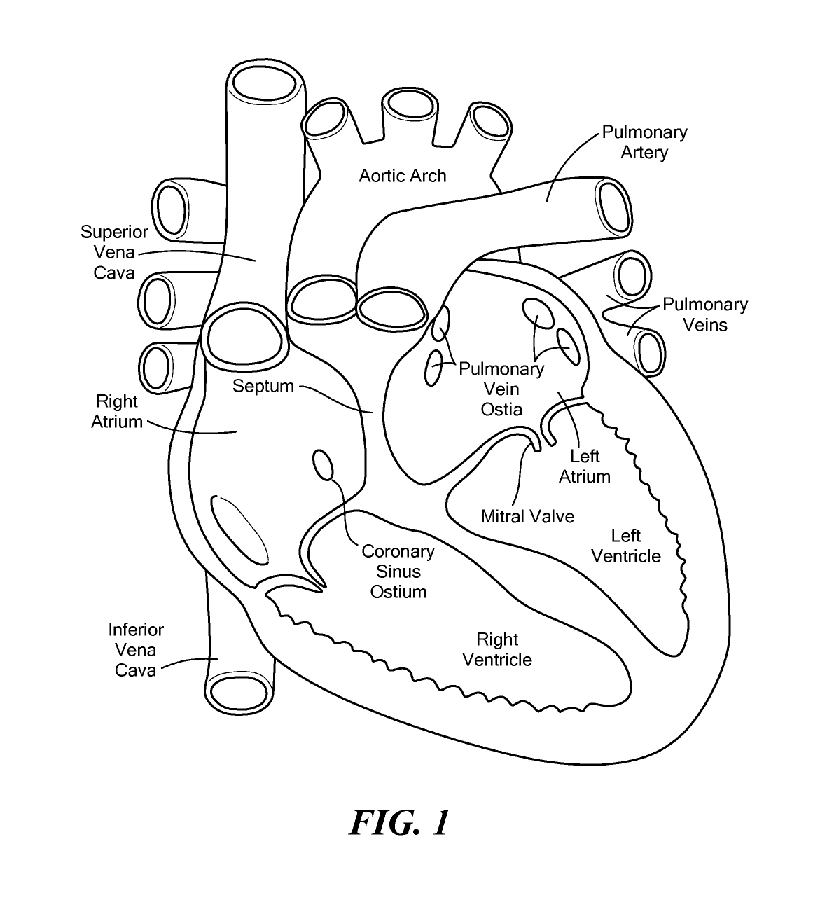

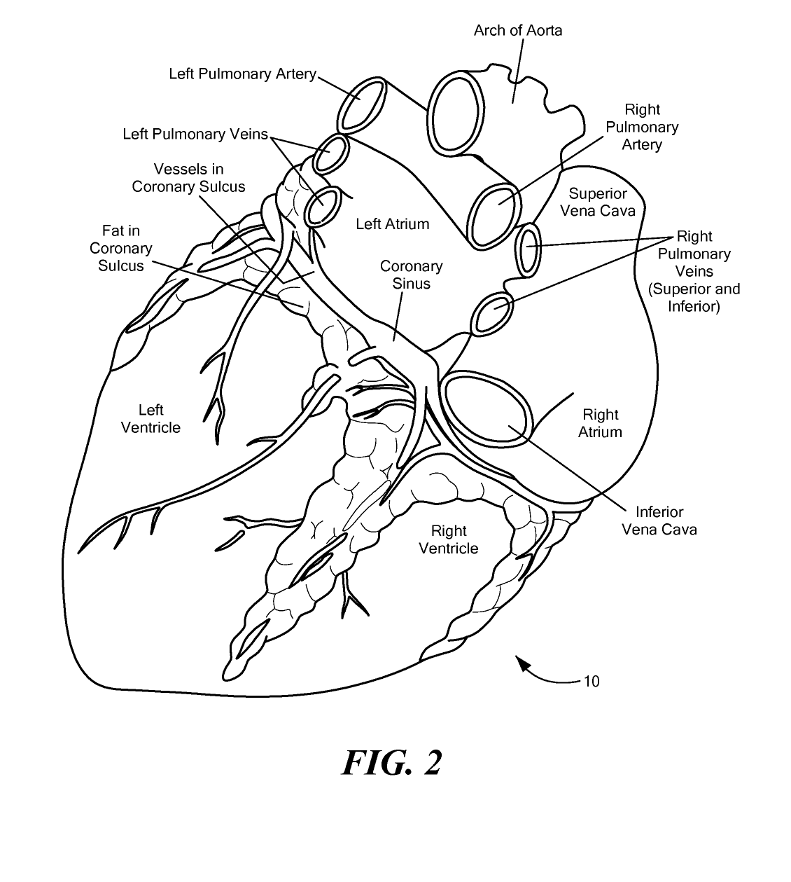

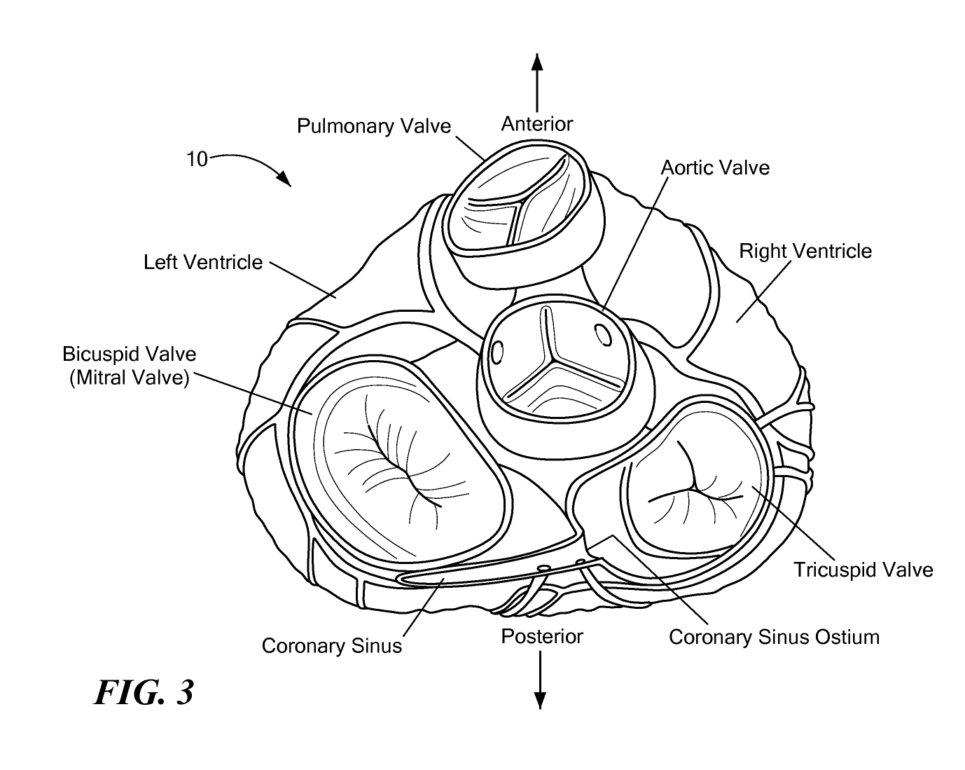

Referring now to the figures, a method, system, and device are shown for creating transmural lesions between the coronary sinus and the left atrium proximate the mitral valve annulus. Referring now to FIGS. 1-3, an anterior view, a posterior view, and a top view of the heart are shown. The coronary sinus is a vein that collects blood from the myocardium and delivers deoxygenated blood to the right atrium of the heart 10. The coronary sinus opens into the right atrium and extends along the coronary sulcus on the posterior side of the heart, proximate the mitral valve annulus. Further, the coronary sinus includes circumferential musculature extending longitudinally along the vein and myocardial fibers that extend obliquely to the inferior wall of the left atrium.

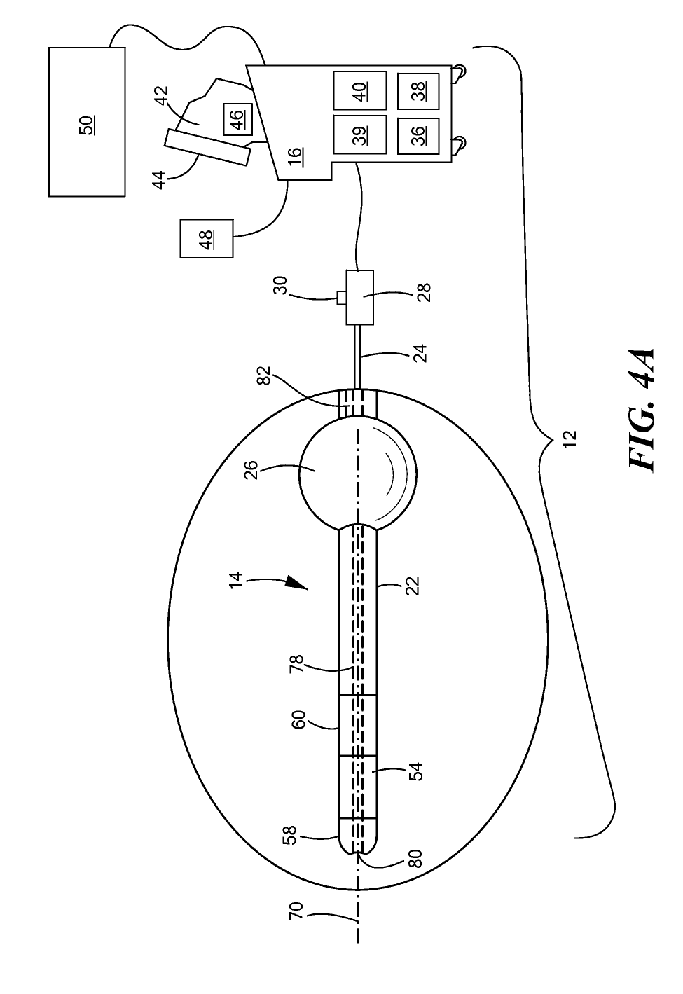

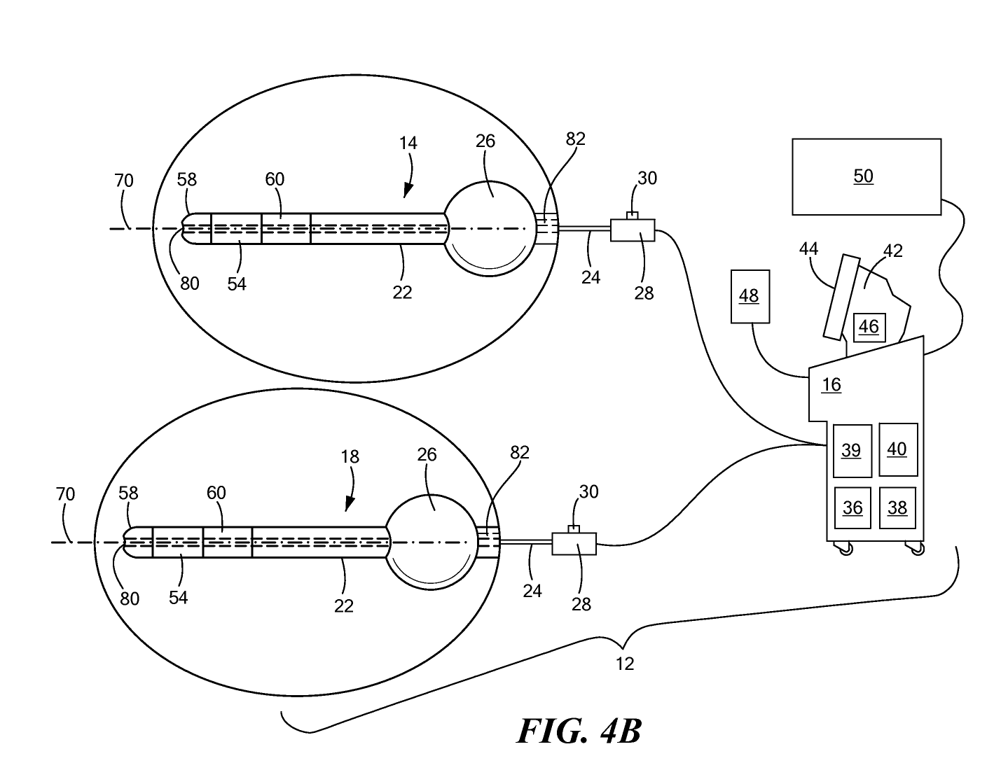

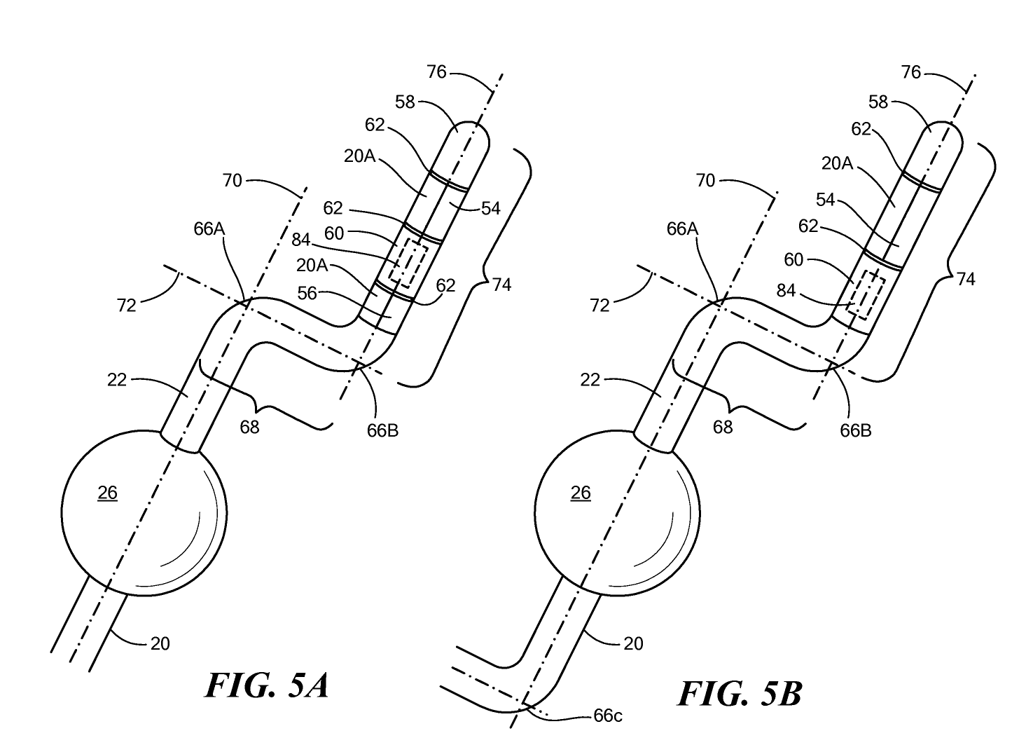

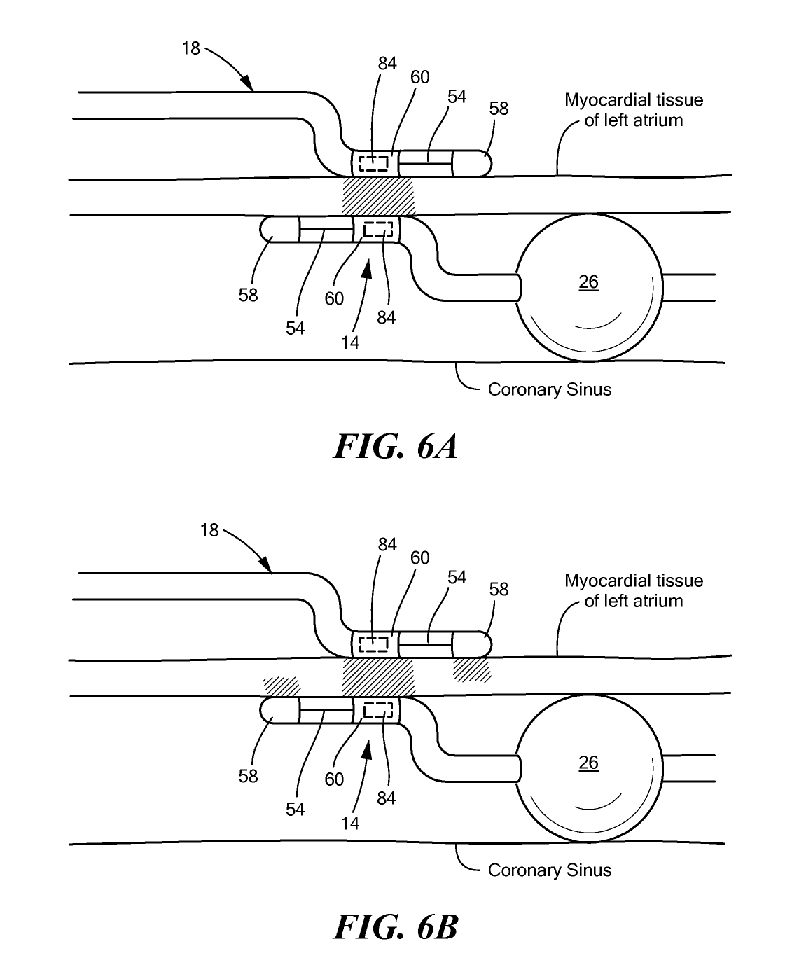

Referring now to FIGS. 4A-5, an ablation system in accordance with the present invention is shown. The system 12 may generally include an ablation catheter 14 and a control unit 16. In one embodiment, the system 12 may include one ablation catheter 14 (as shown in FIG. 4A), and in another embodiment, the system 12 may include a first ablation catheter 14 and a second ablation catheter 18 (as shown in FIG. 4B). Additionally or alternatively, the system 12 may include one or more secondary medical devices for performing various aspects of the medical procedure, such as creating a trans-septal puncture, creating a circumferential ablation lesion around one or more pulmonary veins, mapping cardiac tissue, or the like (not shown). In an embodiment in which the system 12 may be used for mapping cardiac tissue, the system 12 may further include a cardiac signal processing system for receiving and processing mapping data from one or both catheters 14, 18 and/or from secondary medical devices.

Each ablation catheter 14, 18 may be adapted for use with any of a variety of energy modalities, including but not limited to radiofrequency energy, cryotreatment, and electroporation. Further, each ablation catheter 14, 18 may include an elongate body 20 having a distal portion 22 and a proximal portion 24, an occlusion element 26 coupled to the distal portion 22 of the elongate body 20, one or more markers, and one or more ablation electrodes. For example, the occlusion element may be an occlusion balloon 26. The elongate body proximal portion 24 may be coupled to a handle 28 that includes one or more actuation mechanisms 30 (such as knobs, buttons, rings, or the like) for steering the distal portion 22 of the elongate body 20, actuating the one or more electrodes, inflating the occlusion balloon 26, and/or performing other catheter operations.

The control unit 16 may generally include all of the system components, other than the ablation catheters 14, 18, that are used to control, activate, navigate, or transmit and/or receive data from the catheters 14, 18. For example, the control unit 16 may include one or more umbilicals, one or more energy sources 36 (such as a radio frequency energy generator for delivering radio frequency energy and/or AC/DC electroporation energy), a fluid reservoir 38 containing fluid for inflating the occlusion balloon 26 (such as saline), a fluid reservoir 39 for performing a treatment procedure (such as a refrigerant for cryoablation or cryotreatment), a source of contrast fluid 40, and one or more computers 42 having one or more displays 44, one or more processors 46, and one or more user input devices 48. The system 12 may also include equipment for imaging the catheter 14, 18, such as a fluoroscopic imaging system 50 that includes, for example, an X-ray source and fluorescent screen.

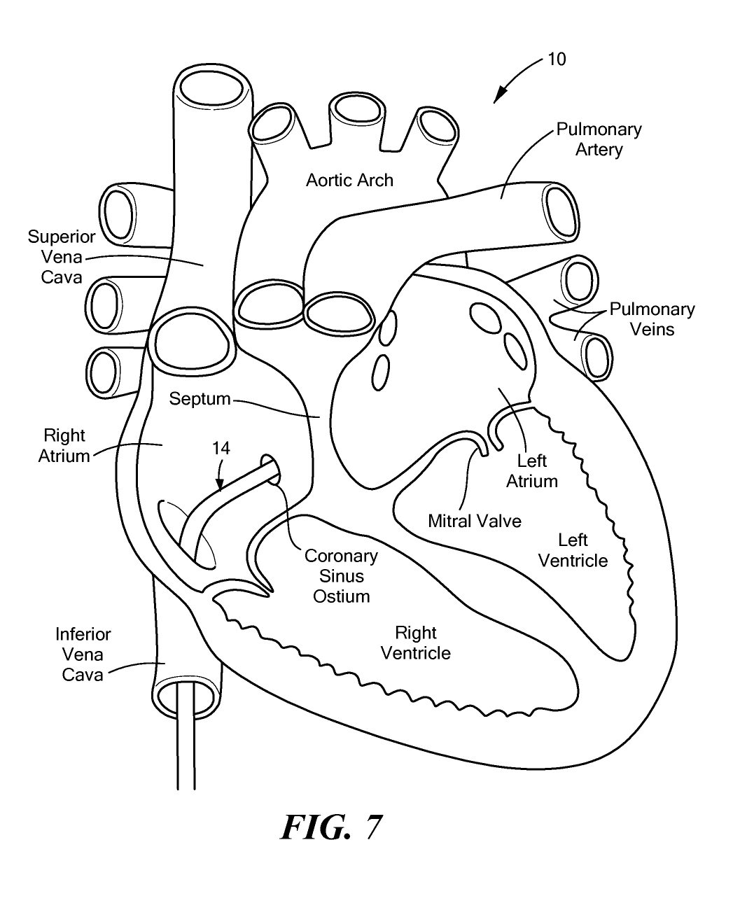

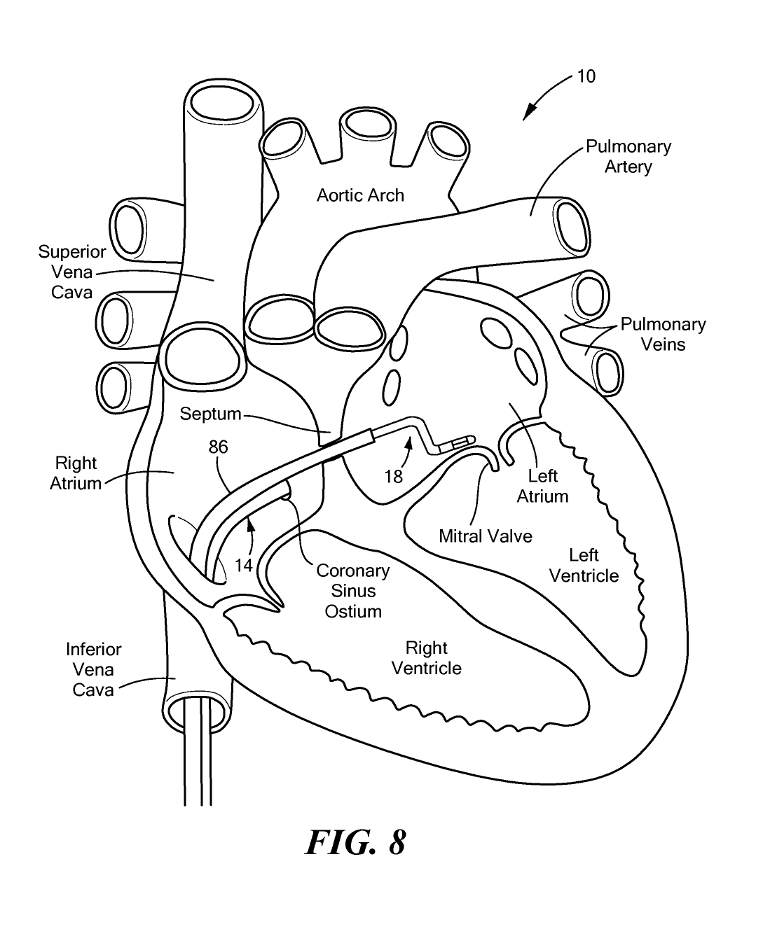

Referring now to FIGS. 5A, 5B, and 6, the ablation catheters 14, 18 are shown and described in more detail. As noted above, each catheter 14, 18 may include one or more markers. For example, each catheter 14, 18 may include a radiopaque hemisphere marker 54 that covers only a portion of the circumference of the elongate body 20 (for example, as shown in FIG. 5B) in order to aid the user in determining the rotational orientation of the catheter 14, 18 when viewed using fluoroscopic imaging. Optionally, the catheter 14, 18 may include a second radiopaque hemisphere marker 56 immediately proximal to an electrode 60 that also covers only a portion of the circumference of the elongate body 20 (for example, as shown in FIG. 5A). Like the first marker 54, the second marker 56 may also indicate the position of the electrode 60. As shown in the figures, for example, the hemisphere marker 54 (and marker 56) may cover approximately half the circumference of the elongate body 20 (that is, approximately 180°±15°. This configuration may enable the user to determine the direction of the catheter 14, 18 and the surface of the elongate body 20 that is in contact with tissue. The one or more portions of the elongate body proximal to but not covered by the hemisphere marker 54 and/or hemisphere marker 56 are indicated in FIGS. 5A and 5B as 20A.

Also as noted above, each catheter 14, 18 may include one or more electrodes. For example, each catheter 14, 18 may include a tip electrode 58 located on the distal tip of the elongate body 20 and a wide-area band electrode 60 that circumscribes the elongate body 20. However, it will be understood that the electrodes 58, 60 may have other configurations. For example, electrode 60 may be on only a portion of the circumference of the elongate body 20 or may include a plurality of discrete electrodes spaced symmetrically or asymmetrically in a partial or complete band around the elongate body. The band electrode 60 may be located proximal to the hemisphere marker 54. For example, the band electrode 60 may be located immediately proximal to the hemisphere marker 54, as shown in FIGS. 5 and 6. However, each catheter 14, 18 (and in both embodiments shown in FIGS. 5A and 5B) may include an insulative section 62 between the hemisphere marker 54 and the tip electrode 58 and the band electrode 60 and, optionally, between the hemisphere marker 56 and the band electrode 60. The markers 54, 56, tip electrode 58, and band electrode 60 may each be composed of an electrically conductive material, such as metal, and the insulative sections 62 may help ensure they are electrically isolated from each other. So, although the electrodes 58, 60 may be referred to as being “immediately distal” or “immediately proximal” to a marker 54, 56, it will be understood that they may be separated by an insulative section 62. Each insulative section 62 may be just thick enough to electrically isolate the electrodes 58, 60 from the one or more markers 54, 56.

Although the electrodes 58, 60 may be in electrical communication with an energy source 36 for delivering radiofrequency, electroporation, and/or other energy to tissue, one or both of the electrodes 58, 60 of the first catheter 14 and/or the second catheter 18 may alternatively be in thermal communication with refrigerant delivered to the distal portion of the elongate body 20 from a fluid reservoir 39. In such a configuration, the electrodes 58, 60 may be thermally transmissive areas. For example, the elongate body 20 may include a fluid delivery lumen (not shown) that delivers refrigerant to a portion of the elongate body that is proximate at least one of the electrodes 58, 60, such that the refrigerant cools the one or more electrodes 58, 60 used for a cryoablation or cryotreatment procedure to a temperature sufficient to cryoablate or cryotreat tissue. As a non-limiting example, the distal tip electrode 58 may be in electrical communication with the energy source and the band electrode 60 may be in fluid communication with the refrigerant reservoir 39 for use of both cryoablation and radio frequency and/or electroporation energy by the same device.

The elongate body 20 may further include a proximal area of deflection 66A and a distal area of deflection 66B (which may also be referred to as deflection joints). Optionally, the elongate body 20 may further include a second proximal area of deflection 66C that is proximal to the balloon 26. Deflection at both of these areas 66A, 66B may create a transverse portion 68 of the elongate body 20 that is at least substantially orthogonal to the longitudinal axis 70 of the catheter 14, 18. However, it will be understood that the elongate body 20 may be deflected at these areas 66A, 66B at an angle that is greater to or less than approximately 90°. Further, deflection at both of these areas 66A, 66B may create a distal tip portion 74 of the elongate body 20 that defines a longitudinal axis 76 that is not coaxial with, but at least substantially parallel to, the catheter longitudinal axis 70. That is, the distal tip portion 74 may be at least substantially parallel to the remaining part of the distal portion of the elongate body 20, excluding the transverse portion 68. The distal tip portion 74 may be distal to the second area of deflection 66B and the transverse portion 68. The width of the transverse portion 68 (that is, the distance between the elongate body 20 and the distal tip portion 74 when an approximately right-angle bend is created at the deflection areas 66A and 66B) may be such that the electrodes 58, 60 may be positioned on the inner wall of the coronary sinus adjacent to the mitral valve annulus. As a non-limiting example, the width of the transverse portion 68 may be approximately 20 mm. However, it will be understood that the width may be greater than or less than approximately 20 mm in order to accommodate patients having a coronary sinus with a smaller or larger inner diameter.

Similarly, deflection at the second proximal deflection area 66C may create a bend in the elongate body 20 proximal to the occlusion balloon 26 (for example, a right-angle bend, although the angle created at the bend may be more or less than approximately 90°). Including the second proximal area of deflection 66C may allow the distal portion of the device to be more precisely maneuvered into the coronary sinus ostium or navigate within the area of the endocardial left atrium.

The catheter 14, 18 may further include one or more pull wires, shims, rods, or other steering mechanisms that are in mechanical communication with the one or more actuation mechanisms 30 of the handle 28. The steering mechanisms may be configured to as to produce a bend at each of the deflection areas 66A, 66B when a force is exerted on the steering mechanisms by the one or more actuation mechanisms 30. As a non-limiting example, one or more pull wires or coils may be anchored to the distal portion of the catheter 14, 18 and used to cause the desired deflections of the elongate body 20. Alternatively, the catheter 14, 18 may be preshaped to naturally assume a deflected configuration when located within the coronary sinus. Thus, the distal portion 22 of the elongate body 20 may be transitionable between an at least substantially linear first configuration (as shown in FIGS. 4A and 4B) and a second configuration in which the elongate body 20 is bent at approximately 90° at each of the deflection areas 66A, 66B (as shown in FIGS. 5 and 6). This transition may be through either manual deflection using one or more steering mechanisms or by virtue of a preshaped configuration. Thus, as used herein, the term “deflection area” may include an area of the elongate body 20 in which a bend may be created, through either manual deflection or by virtue of a preshaped configuration. As a non-limiting example, each of the deflection areas 66A, 66B may be transitionable between an at least substantially linear first configuration and a second configuration in which the first deflection area 66A is deflected at an angle of approximately 90° (for example, 90°±15°) relative to the catheter longitudinal axis 70 and the second deflection area 66B is deflected at an angle of approximately 90° (for example, 90°±15°) relative to the longitudinal axis 72 defined by the transverse portion 68 of the elongate body 20. If the catheter 14, 18 has a preshaped configuration, advancing the catheter 14, 18 out of a sheath or delivery device, in which the catheter is in an at least substantially linear configuration, may cause the catheter 14, 18 to assume the preshaped deflected configuration, for example, as shown in FIGS. 5A-6.

The elongate body 20 may further include or define a contrast fluid injection lumen 78 and the distal tip portion 74 of the elongate body 20 may define a lumen opening 80 for delivering contrast fluid to the external environment when the catheter 14, 18 is used with fluoroscopic imaging (for example, as shown in FIG. 1). The fluid injection lumen 78 may be in fluid communication with the contrast fluid source 40. If two ablation catheters 14, 18 are used, only the catheter 14 that is positioned within the coronary sinus may include the contrast injection lumen, lumen opening 80, and occlusion balloon 26, as these components may not be of use on the catheter 18 that is positioned within the left atrium of the heart 10. Alternatively, the second catheters 18 may include all of the same components as the first catheter 14.

The occlusion balloon 26 may be located proximal to the proximal area of deflection 66A. During use, the occlusion balloon 26 may be inflated with fluid from the fluid reservoir 38 (such as through one or more fluid lumens in the elongate body 20) in order to occlude the coronary sinus and prevent blood from passing into the right atrium of the heart 10 and to ensure the contrast fluid remains at the site of the ablation procedure and to help maintain the catheter position. The catheter 14, 18 may also include one or more fluid lumens 82 in communication with the fluid reservoir 38 for the delivery and recovery of fluid to the occlusion balloon 26 (for example, as shown in FIG. 1). The occlusion balloon 26 may be composed of any expandable and flexible material, and may be compliant (such as latex, polyurethane, nylon elastomers, thermoplastic elastomers), semi-compliant (such as polyethylene terephthalate (PET), nylon, polyurethane), or non-compliant (such as PET and nylon). As a non-limiting example, the occlusion balloon 26 may be composed of silicone, which is compliant enough to allow occlusion but is not likely to damage the coronary sinus. Further, the occlusion balloon 26 may be sized to occlude the coronary sinus at a location proximate the coronary sinus ostium leading into the right atrium. The occlusion of the coronary sinus may retain contrast fluid for improved imaging of the coronary sinus anatomy and may also stabilize the catheter. It may also help improve the effectiveness of the ablation by reducing the cooling effect of blood flow in the coronary sinus. The coronary sinus may have a larger inner diameter proximate the right atrium, and the occlusion balloon 26 may have a maximum diameter that is sufficient to occlude this portion of the coronary sinus. However, the occlusion balloon 26 may have an adjustable diameter (for example, depending on the amount of inflation fluid injected into the balloon 26) such that the occlusion balloon 26 may be sized to occlude portions of the coronary sinus having a smaller inner diameter. To achieve this, the system 12 may include one or more valves or fluid flow regulation devices (not shown) to control the delivery of fluid from the fluid reservoir 38 to the occlusion balloon 26. When two ablation catheters 14, 18 are used, the catheter 18 positioned in the left atrium and used to ablate the posterior wall of the endocardial left atrium mitral isthmus proximate the mitral valve annulus may not include a balloon 26. Alternatively, the catheter 18 may include an occlusion balloon 26, but the occlusion balloon 26 may not be inflated during the procedure.

Each ablation catheter 14, 18 may further include a magnet 84 within the distal tip portion 74 of the elongate body 20. For example, the magnet 84 may be located within the distal tip portion 74 proximate the band electrode 60. However, it will be understood that the magnet 84 may be located in another suitable location, such as proximate the hemisphere marker 54 or the tip electrode 58. Additionally, the magnet may be on an outer surface of the elongate body 20 or within the elongate body 20. As shown in FIGS. 6A and 6B, the magnets 84 may cause the catheters 14, 18 to be magnetically attracted to each other, thereby facilitating placement of the catheters 14, 18 at the target treatment site. As shown, for example, the first catheter 14 may be positioned within the coronary sinus and the second catheter 18 may be positioned at the posterior wall of the left atrium, proximate the mitral valve annulus. This magnetic coupling of the catheters through the tissue of the coronary sinus and the left atrium may enable the catheters 14, 18 to together create a transmural lesion (shown in FIGS. 6A and 6B with diagonal lines between the electrodes 60 of each catheter 14, 18) between the left atrium and coronary sinus. The tip electrode 58 and/or the band electrode 60 of each catheter 14, 18 may deliver energy, for example, radiofrequency energy in unipolar mode and/or bipolar mode, to create a transmural lesion between the two catheters 14, 18. Additionally or alternatively, electroporation and/or cryoablation may be used to create the transmural lesion. The magnets 84 may each exert a magnetic field that is strong enough to magnetically couple the two catheters 14, 18 through biological tissue, such as coronary sinus and atrial wall tissue.

Further, the distal tip electrode 58 of the first catheter 14 may be used to create a second tissue lesion and the distal tip electrode 58 of the second catheter 18 may be used to create a third tissue lesion (for example, as shown in FIG. 6B). For example, unipolar energy may be delivered by the distal tip electrodes 58. The lesions created by the distal tip electrodes 58 may or may not be transmural lesions, although shown as being non-transmural in FIG. 6B.

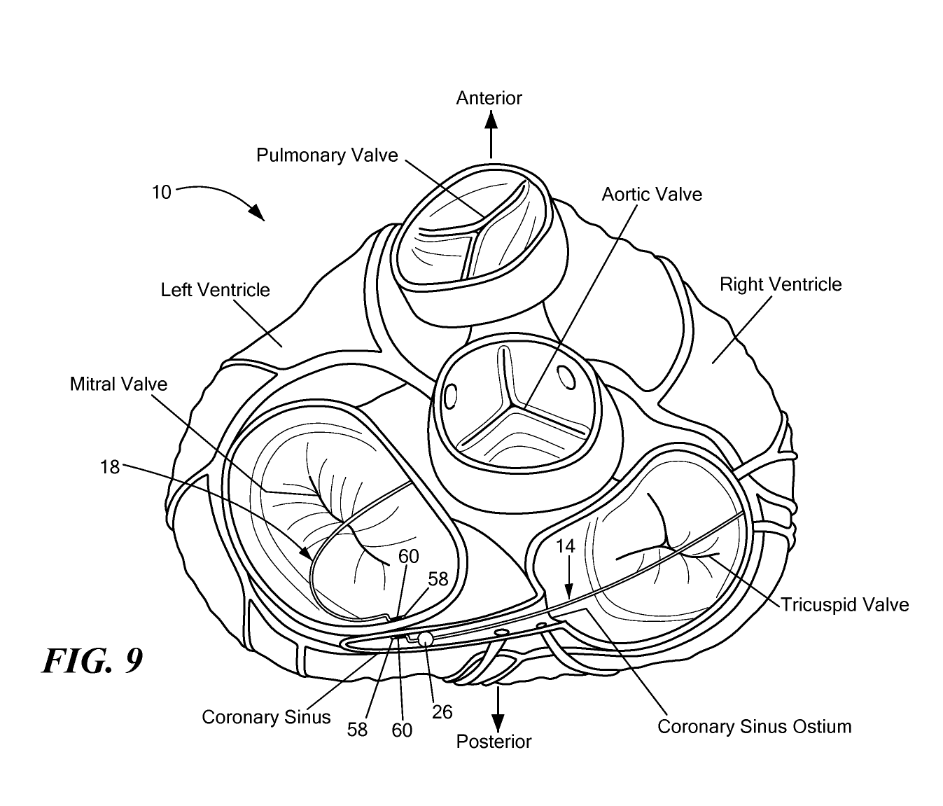

Referring now to FIGS. 7-9, a method of positioning the first ablation catheter and second ablation catheter proximate each other is shown. As a non-limiting embodiment, a first ablation catheter 14 may be navigated through the vasculature of a patient to the inferior vena cava and into the right atrium of the heart 10. From the right atrium, the catheter 14 may then be navigated through the coronary sinus ostium and into the coronary sinus on the posterior side of the heart 10. The first catheter 14 may be positioned at a location within the coronary sinus at which the tip electrode 58 and/or the band electrode 60 are proximate the mitral valve annulus, located within the left atrium. The occlusion balloon 26 may be inflated with fluid from the fluid reservoir 38 to a diameter sufficient to occlude the coronary sinus. When the first ablation catheter 14 is at the target treatment site, the distal tip portion 74 of the elongate body may be located further within the coronary sinus than the occlusion balloon 26, which may be located closer to the coronary sinus ostium.

A guide sheath 86 or similar delivery device may likewise be navigated through the patient's vasculature to the inferior vena cava and into the right atrium of the heart 10. From the right atrium, a puncture device (or the guide sheath 86, if so configured) may be used to create a trans-septal puncture, creating a passage through the fossa ovalis between the right atrium and left atrium through which the guide sheath 86 may be passed. A second ablation catheter 18 may be navigated through the guide sheath 86 and into the left atrium. Once within the left atrium, the second catheter 18 may be navigated to the target treatment site, for example, the posterior wall of the endocardial left atrium at the mitral isthmus proximate the mitral valve annulus. It will be understood that either the first 14 or second 18 ablation catheter may be positioned first (that is, the second ablation catheter 18 may be positioned within the left atrium before the first ablation catheter 14 is positioned within the coronary sinus) or they may be positioned simultaneously.

Once both ablation catheters 14, 18 are positioned proximate each other on opposite sides of the left atrial wall, the magnets 84 within both catheters 14, 18 may cause the catheters 14, 18 to become magnetically coupled to each other as described above. Once the catheters 14, 18 are magnetically coupled at the target treatment site, the energy source 36 may be activated to transmit radiofrequency energy to the tip electrodes 58 and/or the band electrodes 60. The tip electrodes 58 and/or the band electrodes 60 may deliver radiofrequency energy to contacted tissue in unipolar mode, the combination of unipolar and/or bipolar energy from both catheters 14, 18 creating a transmural lesion. Energy may be delivered as an ablative waveform resulting in irreversible electroporation of the myocytes, as duty-cycled radiofrequency energy, unipolar radiofrequency energy, and/or bipolar radiofrequency energy. Additionally or alternatively, refrigerant may delivered to at least one electrode 58, 60 of at least one of the catheters 14, 18. The magnetic coupling of the catheters 14, 18 may facilitate placement of the catheters 14, 18 at the target treatment site, prevent movement of the catheters 14, 18 away from the target treatment site, and facilitate the creation of a transmural lesion between the left atrium and the coronary sinus. Further, the distal tip electrode 58 of the first catheter 14 may be used to create a second tissue lesion and the distal tip electrode 58 of the second catheter 18 may be used to create a third tissue lesion.

The proximity of the electrodes of the catheters 14, 18 made possible by the magnetic coupling may create transmural lesions by either the transmission of energy between the catheters 14, 18 through the intervening tissue (for example, when radiofrequency and/or electroporation energy is used) or may allow a lesion created by each catheter 14, 18 to overlap each other and create a single transmural lesion (for example, when radiofrequency, electroporation, and/or cryoablation is used as an energy modality). A transmural lesion may be more effective at treating atrial fibrillation and atrial flutter than a non-transmural lesion.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. An ablation device comprising:
an elongate body defining a longitudinal axis and having a distal portion, a proximal portion, a first deflection area in the distal portion, and a second deflection area in the distal portion;
at least one ablation electrode at the distal portion; and
a magnet at the distal portion.

2. The ablation device of claim 1, wherein the ablation device includes a first electrode and a second electrode, the first electrode being distal to the second electrode.

3. The ablation device of claim 2, wherein the first electrode is a distal tip electrode and the second electrode is a band electrode.

4. The ablation device of claim 3, wherein the band electrode is located proximal to the distal tip electrode.

5. The ablation device of claim 2, further comprising at least one radiopaque marker.

6. The ablation device of claim 5, wherein the device includes one radiopaque marker that covers a portion of a circumference of the elongate body at a location between the first electrode and the second electrode.

7. The ablation device of claim 5, wherein the device includes a first radiopaque marker that covers first portion of a first circumference of the elongate body at a location between the first electrode and the second electrode, the device further including a second radiopaque marker that covers a portion of a second circumference of the elongate body at a location proximal to the second electrode.

8. The ablation device of claim 6, wherein the radiopaque marker covers approximately half the circumference of the elongate body.

9. The ablation device of claim 1, further comprising an occlusion element coupled to the distal portion of the elongate body.

10. The ablation device of claim 9, wherein the occlusion element is coupled to the distal portion of the elongate body at a location proximal to the first and second deflection areas.

11. The ablation device of claim 10, further comprising a third deflection area in the distal portion, the third deflection area being proximal to the to occlusion element.

12. The ablation device of claim 1, wherein the elongate body is transitionable between an at least substantially linear first configuration and a second configuration in which the distal portion of the elongate body includes a transverse portion that is substantially orthogonal to the longitudinal axis of the elongate body and a distal tip portion that defines a longitudinal axis that is non-coaxial with and parallel to the longitudinal axis of the elongate body.

13. The ablation device of claim 11, wherein the transverse portion defines a longitudinal axis, the first deflection area being deflected approximately 90° from the longitudinal axis of the elongate body and the second deflection areas being deflected approximately 90° from the longitudinal axis of the transverse portion when the elongate body is in the second configuration.

14. The ablation device of claim 3, wherein the magnet is located within the distal portion of the elongate body proximate the band electrode.

15. An ablation system, the system comprising:
a first ablation device and a second ablation device, each of the first and second ablation devices including:
an elongate body defining a distal tip and a longitudinal axis and having a distal portion, a proximal portion, a first deflection area in the distal portion, and a second deflection area in the distal portion;
a first ablation electrode at the elongate body distal tip and a second ablation electrode proximal to the first electrode; and
a magnet at the distal portion, the magnet of the first ablation device and the magnet of the second ablation device being configured to magnetically couple the first and second ablation devices through tissue.

16. The ablation system of claim 15, wherein the first ablation device further includes an occlusion balloon at the distal portion proximal to the second ablation electrode.

17. The ablation system of claim 16, further comprising a control unit in at least one of electrical and mechanical communication with each of the first ablation device and second ablation device.

18. The ablation system of claim 15, further comprising a fluoroscopic imaging system in communication with the control unit.

19. The ablation system of claim 15, wherein the elongate body of the first ablation device includes a fluid injection lumen extending between the proximal portion of the elongate body to the distal portion of the elongate body, the distal tip defining a lumen opening.

20. The ablation system of claim 15, wherein the elongate body of each of the first and second ablation devices is transitionable between an at least substantially linear first configuration and a second configuration in which the distal portion of the elongate body includes a transverse portion that is substantially orthogonal to the longitudinal axis of the elongate body and a distal tip portion that defines a longitudinal axis that is non-coaxial with and parallel to the longitudinal axis of the elongate body.

21. The ablation system of claim 20, wherein the transverse portion defines a longitudinal axis, the first deflection area is deflected approximately 90° from the longitudinal axis of the elongate body and the second deflection areas is deflected approximately 90° from the longitudinal axis of the transverse portion when the elongate body is in the second configuration.

22. The ablation system of claim 15, each of the first and second ablation devices further including a radiopaque marker covering approximately half a circumference of the elongate body and being located between the first electrode and the second ablation electrode.

23. The ablation system of claim 22, the radiopaque marker being a first radiopaque marker and the circumference is a first circumference, each of the first and second ablation devices further including a second radiopaque marker covering approximately have a second circumference of the elongate body and being located proximal to the occlusion balloon.

24. The ablation system of claim 15, wherein the control unit includes at least one of a radiofrequency energy source in electrical communication with the first and second electrodes and a fluid source in fluid communication with the occlusion balloon.

25. The ablation system of claim 15, wherein the control unit includes a refrigerant reservoir and at least one of the first ablation device and the second ablation device includes a fluid delivery lumen in fluid communication with the refrigerant reservoir, the fluid delivery lumen being in thermal communication with the first and second electrodes.

26. A method of treating cardiac arrhythmia, the method comprising:
positioning a first ablation catheter in a coronary sinus proximate an endocardial left atrial mitral isthmus of a heart, the first ablation catheter including:
an elongate body defining a distal tip and a longitudinal axis and having a distal portion, a proximal portion, a first deflection area in the distal portion, and a second deflection area in the distal portion;
at least one ablation electrode on the distal portion of the elongate body;
an occlusion balloon on the distal portion proximal to the first deflection portion; and
a magnet at the distal portion distal to the second deflection portion;
positioning a second ablation catheter within a left atrium of the heart proximate the endocardial left atrial mitral isthmus, the second ablation catheter including:
an elongate body defining a distal tip and a longitudinal axis and having a distal portion, a proximal portion, a first deflection area in the distal portion, and a second deflection area in the distal portion;
at least one ablation electrode on the distal portion of the elongate body; and
a magnet at the distal portion distal to the second deflection portion,
the magnet of the second ablation catheter being proximate the magnet of the first ablation catheter;
magnetically coupling the first and second ablation catheters through tissue of the coronary sinus and left atrium;
activating the at least one electrode of each of the first and second ablation catheters; and
creating a transmural lesion in the tissue between the at least one electrode of the first ablation catheter and the at least one electrode of the second ablation catheter.

27. The method of claim 26, wherein activating the at least one electrode includes delivering at least one of radiofrequency energy and electroporation energy to the at least one electrode.

28. The method of claim 26, wherein activating the at least one electrode includes circulating a refrigerant within at least the distal portion of each of first and second ablation catheters.

29. The method of claim 26, wherein the elongate body of each of the first and second ablation catheters is transitionable between an at least substantially linear first configuration and a second configuration in which the distal portion of the elongate body includes a transverse portion that is substantially orthogonal to the longitudinal axis of the elongate body and a distal tip portion that defines a longitudinal axis that is non-coaxial with and parallel to the longitudinal axis of the elongate body.
